(19) United States
(12) Patent Application Publication
(10) Pub. No.: US 2007/0167705 A1
(43) Pub. Date: Jul. 19, 2007

(54) Integrated ultrasound imaging system

(76) Inventors: Alice M. Chiang, Weston, MA (US); Albert S. Kyle, Andover, MA (US); Michael Brodsky, Brookline, MA (US)

Correspondence Address: WEINGARTEN, SCHURGIN, GAGNEBIN & LEBOVICI LLP, TEN POST OFFICE SQUARE, BOSTON, MA, 02109, US

(21) Appl. No.: 11/498,316

(22) Filed: Aug. 2, 2006

Related U.S. Application Data
(60) Provisional application No. 60/705,542, filed on Aug. 4, 2005.

(51) Int. Cl. A61B 5/05

(52) U.S. Cl. 600/407; 600/411

(57) ABSTRACT

The present invention relates to systems and methods for performing integrated ultrasound and magnetic resonance imaging procedures on a patient. An ultrasound probe is used within an MRI system to provide both imaging modalities for diagnostic and therapeutic applications.

### CROSS REFERENCE TO RELATED APPLICATION

This application claims the priority of U.S. Provisional Application No. 60/705,542, filed Aug. 4, 2005 entitled, INTEGRATED ULTRASOUND IMAGING SYSTEM. The entire content of the above application is being incorporated herein by reference.

### BACKGROUND OF THE INVENTION

Magnetic resonance imaging (MRI) and real time ultrasound diagnostic imaging are common medical procedures, routinely ordered to diagnose a variety of disorders of the abdomen, heart, brain, vasculature and other parts of the body. When both procedures are ordered, MRI and ultrasound images are collected at separate times and locations, reviewed by separate physicians, and then reported to a third physician for review and synthesis with other relevant information. MRI uses a magnetic and RF coil system to provide a controllable electromagnetic filed to image a region of interest The use of electrical and other components near the MRI system can interfere with normal imaging procedures.

MRI and ultrasound are synergistic and complementary modalities. Both are non-invasive and non-ionizing; each has its own respective strengths, weaknesses, costs and benefits. MRI has superior spatial resolution and a large field of view; ultrasound has superior temporal resolution and in many applications has a lower cost when compared with MRI. Both modalities can be reconstructed into three dimensional (3D) images after acquisition, which may be important when characterization of complex anatomical structures is important for an accurate diagnosis. In some cases, 3D cardiac ultrasound imaging is done in real time.

A continuing need exists for further improvements in the use of MRI and ultrasound imaging systems to provide for a more complete diagnostic capability.

### SUMMARY OF THE INVENTION

The present invention relates to systems and methods for performing integrated MRI and ultrasound imaging. A preferred embodiment of the invention provides an ultrasound probe assembly that can be used in proximity with an MRI system. A single control or timing system can be used to gate ultrasound and MRI acquisition periods. It is often desirable to sequentially acquire the two images at different times so as to minimize or eliminate interference between the two imaging sequences.

Co-registration of MRI and ultrasound images can facilitate the image review process. In a preferred embodiment, ultrasound data is acquired using one coordinate system, MRI data is acquired using a second coordinate system, and a registration method is used to overlay the two images, one on the other, so that they can be displayed simultaneously on a single display device. This allows the reviewing physician to visually integrate the separately acquired images. Because the collection of the respective images can be done at different times, post processing registration can produce anomalies both in position and time. For this reason, as well as the additional time required to post process the images, co-registration of MRI and ultrasound for review is not performed in the clinic. Thus, a preferred embodiment of the present invention includes co-registration of MRI and ultrasound images.

Pediatric cardiology is one example of the strengths and weaknesses of MRI and ultrasound imaging. Pediatric cardiologists often need to display high resolution 3D image data of a congenitally malformed pediatric heart to allow visualization of complex cardiac anatomy. Although it is possible to display the pediatric heart using 3D ultrasound, the superior spatial resolution of MRI makes it the preferred method to view fine detail. However the relatively high heart rates common in pediatric patients requires the use of ultrasound systems capable of high frame rate imaging. In these cases, neither MRI nor ultrasound alone may be able to do the job.

Efforts to improve ultrasound and MRI systems to overcome their respective disadvantages have not been very successful. Current 3D ultrasound systems can achieve real-time 3d imaging at the expense of degraded spatial and temporal resolution. ultrasound systems in 2D mode can achieve better spatial resolution at frame rates as high as 50-100 frames/second, but have a small field of view represented by a very narrow sector width. Neither 2D nor 3d ultrasound can compare with the anatomical detail of a cardiac MRI image combined with its large field of view. Cardiac MR Angiography (CMRA) suffers from low frame rates (typically 4 frames/second) that are inadequate to study the beating heart. Additionally, CMRA is challenged with data acquisition problems that relate to motion artifacts and timing the sequence of each slice.

A preferred embodiment of the invention further uses the MRI imaging system for the imaging of tissue or organs undergoing ultrasound treatment procedures. Treatment methods such as high intensity focused ultrasound (HIFU) for tumor thermal treatment can be used with simultaneous or sequential MRI data acquisition to monitor proper focusing and temperature of the region of interest.

### BRIEF DESCRIPTION OF THE DRAWINGS

FIG. 1 schematically illustrates a system for MRI and ultrasound imaging in accordance with a preferred embodiment of the invention;

FIGS. 2A-2C illustrates a triggering system for an integrated ultrasound and MRI system;

FIG. 3 illustrates a timing diagram for synchronous simultaneous data acquisition;

FIG. 4 illustrates a timing diagram for synchronous gated data acquisition;

FIG. 5 schematically illustrates a control system used for integrated ultrasound and MRI image acquisition;

FIG. 6 illustrates a method for monitoring heart movement and trigger and MRI image.

FIG. 7 illustrates a method for selecting different views of a heart using an ultrasound system; and

FIG. 8 illustrates a method for marking anatomical locations such as the heart for registration of images and marking boundaries.

### DETAILED DESCRIPTION OF THE INVENTION

The present invention relates to systems and methods for combined ultrasound and magnetic resonance sequential imaging. Dynamic spatial organ localization to improve MRI & CMRA images, based on steady-state free precession (SSFP) sequence, can be performed by real-time ultrasound. Timing of the sequence of acquisition of MRI data is currently gated by EKG, respiration and “navigator” signals that monitor motion of structures, such as the diaphragm. In a preferred embodiment, ultrasound is used for improved MRI gating, based on motion detection in the region of interest (ROI). Ultrasound can improve the signal to noise ratio, and result in better overall image quality of the MRI images. Ultrasound can benefit the acquisition of CMRA images. CMRA is a method that holds great promise to provide a comprehensive cardiac exam, including imaging of the coronary arteries. CMRA can avoid ionizing radiation and potentially harmful contrast agents that are required for XRAY based methods, such as coronary angiography and spiral CT. CMRA faces challenges, among which is the problem of movement of the region of interest (ROI) caused by respiration and other patient movement.

A preferred method for an integrated ultrasound system is to predict the position of the coronary arteries to ensure that gating of MRI data acquisition occurs when they are at the same position. This can be done with tissue Doppler imaging (TDI), in a manner similar to a method used for diagnostic testing. Real-time assessment of left ventricular dyssynchrony (LVD) is currently performed with specialized 2D & 3D ultrasound systems. The rate of deformation of the left ventricle indicates the degree of disease, which can originate from a variety of causes, including electrophysiology defects, myocardial infarction or heart failure. LVD diagnosis is highly sensitive to the precise timing during systole (best assessed with high frame rate ultrasound) and accurate measurements of the left ventricle in multiple planes (best assessed with 3D MRA). MRA can enhance the diagnostic accuracy of LVD.

Patient convenience is enhanced by combining two visits into one. Preferably, an integrated system of ultrasound combined with MRI reduces time and cost for both provider and patient as well as use the complementary aspects of the two systems in combination.

Positioning of endoscopic devices, including catheters, at a specific point in the body is normally done with X-ray angiography, but also can be done by MRI and ultrasound. Location can be best resolved by MRI. Detection of the timing of moving parts of the body, particularly the heart, is best determined by ultrasound. Placement of electrophysiology catheters at a precise point in the cardiac cycle (e.g. during the systolic phase) is one example, because of the ability of ultrasound to visualize valve motion and pinpoint beginning and end of systole. The low frame rates of MRI render it inadequate for the timing needs of diagnostic and/or therapeutic procedures in the heart.

Biopsy of breast lesions is performed under image guidance, typically with an X-ray fluoroscopy system. In some cases, accidental puncture of a blood vessel can occur during needle biopsy, because small vessels are difficult to visualize with a fluoroscope. This risk can be avoided by a combination of MRI and ultrasound to provide an accurate image of the breast (MRI) plus flow information as well as endoscopic device guidance using ultrasound. MRI can have increased sensitivity and specificity in the detection of breast lesions, and is preferred to diagnose breast cancer compared with conventional mammography.

Imaging of the brain to prepare for neurosurgery is routinely performed prior to surgery. When surgery begins, the skull is opened and a loss of cerebral spinal fluid (CSF) occurs, causing the brain to move as much as 1 cm—known as “brain shift.” This disturbs the spatial relationships that were established in the pre-op images, and requires real-time adjustments by the surgeon. Real-time tracking of the displacement of the brain may be performed with ultrasound. Similarly, less-invasive neuro-interventional devices require real-time image guidance by the neuro-radiologist.

Therapeutic ultrasound is an important method for a wide variety of treatments, including non-invasive tissue ablation using heat and cavitation, treatment of vessel puncture resulting from endoscopic procedures and/or trauma, and blood brain barrier disruption to enable drug delivery to the brain. MRI guidance of simultaneously delivered therapeutic ultrasound is currently limited by the respective interference of the RF components of each of the two modalities. Simultaneous MRI and ultrasound enable MRI guidance of ultrasound therapy.

Efforts to achieve simultaneous MRI and ultrasound have been confounded by several problems. These may be avoided and/or solved with simultaneous MRI and ultrasound as set forth in the present invention.

The large size of conventional ultrasound systems creates problems to shield the ultrasound system from the magnetic field, and has discouraged the use of ultrasound systems in close proximity to the MRI system. The ultrasound system emits radio frequency (RF) signals that interfere with the MRI system, and vice versa. Both systems are thus susceptible to RF signals from the other so that ultrasound probe and MRI RF coil placement may interfere with each other. Also, the high magnetic field of the MRI system is a challenging environment. The high strength MRI magnetic coils can cause electronic equipment within the field to move in an unpredictable, dangerous manner. Ultrasound system components and transmission cables must be properly shielded to ensure safety of patients and staff. Shielding the ultrasound system includes the use of special materials such as metal-coated Mylar that enclose the transducer housing and the pod or interface housing.

Magnetic resonance imaging (MRI) of moving parts of the body is difficult to perform because of motion artifacts introduced into the MRI image. Various methods have been used to reduce those artifacts. These include signals generated in a region of the body by means of a pulse sequence, known as “navigator signals.” These navigator signals are generated in a manner independent of other spatially encoded MR signals. A Fourier transformation is applied to the navigator signal to obtain an ID proton density profile of the region, and the position of the moving part is then determined.

Other methods for artifact reduction include techniques to derive phase and frequency correction from navigator signals. These derived corrections can be applied to and combined with traditional methods, such as EKG triggering and respiratory gating, The combined signals are used to counteract motion artifacts in magnetic resonance images such as MRI images of the coronary artery. One drawback of the EKG triggering method is that the position derived from the EKG deviates from the actual position of the moving part in the body. This is caused by the relationship between the EKG and the actual, mechanical cardiac motion. An incorrect position of the region to be excited is adjusted in the body, and motion artifacts still occur in the MRI image. It is therefore preferable to use a method that is mechanical and not electrical, as the mechanical motion is not always perfectly timed to the electrical activity of the heart.

Fixation of the ultrasound transducer to the patient during simultaneous MRI and ultrasound is a potential problem. In a typical echocardiography exam, the transducer is held against the chest of the patient by a technician or by the echocardiographer (cardiologist). Then, the transducer can be carefully positioned and manipulated to achieve certain views of the heart that are relevant to the diagnosis being performed. In a combined simultaneous exam, it is difficult for both patient and technician to be present within the magnetic field of the MRI machine. Rather, it is preferable that the ultrasound transducer be positioned prior to the beginning of the MRI procedure and left in place for the duration of the MRI exam. A new method and device is needed to fixate the ultrasound transducer.

A preferred embodiment of the invention is a modular ultrasound system architecture having a front end, a transmission cable and back end computer. The ultrasound front end circuits are enclosed in a small module, that can also be referred to as a “pod.” The transducer or both the pod and ultrasound transducer are placed within the magnetic field, near the patient to allow placement of the transducer on the patient.

The transducer is held against the body, for example the chest of the patient by a fixation device and method. In one embodiment of the fixation device and method, the patient lies on his/her stomach, and the transducer is placed beneath the patient in conjunction with a body support device such as a movable MRI table that supports the patient during the MRI procedure. In this embodiment, a technician carefully positions the transducer prior to the MRI scan, and the weight of the patient holds the transducer in place during the MRI scan. In another embodiment, the patient lies on his/her back, and the fixation device holds the transducer in place with a body mounting device such as a harness, corset, spring, strap, adhesive, or other fixation method. In both embodiments, either an existing transducer probe is shielded to allow operation within the magnetic field, or a transducer is used without ferrous materials and shielding requirements. Alternatively, the cable connecting the transducer to the pod can be increased in length to permit a portion of the transducer to be within the magnetic field, and another portion (the connector for example) to be located outside the magnetic field. Both embodiments include the possibility of use of electromechanical systems to remotely control, position and adjust the transducer probe during the MRI scan, to augment the manual manipulation of the transducer probe by a technician.

The computer is located outside the magnetic field, at a distance of about 100 ft (30 meters) or more from the pod and transducer. Shielding is facilitated by the small size of the pod/transducer combination, and is not required for the computer. Transmission of timing RF signals between ultrasound and MRI systems is accomplished via a standard interface channel, such as TTL. A fiber optic cable connects the pod to a computer containing the “back end” signal processing circuits. All ultrasound system elements are heavily shielded to avoid interference from RF and magnetic signals.

The proposed method is the use of timing of the radio frequency (RF) signals of the MRI and ultrasound systems that ensures that the receivers of each system are timed to avoid reception of the RF signal of the other system. RF signal timing can also be augmented with gating techniques, described as follows.

Tissue Doppler Imaging (TDI) is a method that can be used to accomplish the goal of artifact reduction in moving MRI images. TDI can be used separately, or in combination with navigator signals, phase and frequency derived navigator signals, EKG triggering and respirator gating to accomplish the artifact reduction desired affect.

One implementation of TDI is a method for rapid, accurate, quantitative evaluation of cardiac function and ventricular wall motion (dyssynchrony) using Triplex mode TDI. Using this method, analyzing multi-gate pulsed wave spectral Tissue Doppler data, strain images and strain-rate analysis can be provided. This is a rapid, accurate method for real-time evaluation of cardiac function and identification of ventricular wall motion dyssynchrony using quantitative analysis of B-mode images. An automatic border detection (ABD) technique can delineate and track the movement of left ventricular endocardial contours. The use of TDI as described above, used separately, or when combined with EKG triggering, respiratory gating and conventional navigator signals can reduce MRI artifacts.

One implementation of the method is to place one Doppler cursor on a landmark fixed within the magnetic field, and the other Doppler cursor on the moving heart. Then, the actual mechanical motion can be assessed directly from the TDI signal, thus avoiding problems of linking the EKG signal to actual motion.

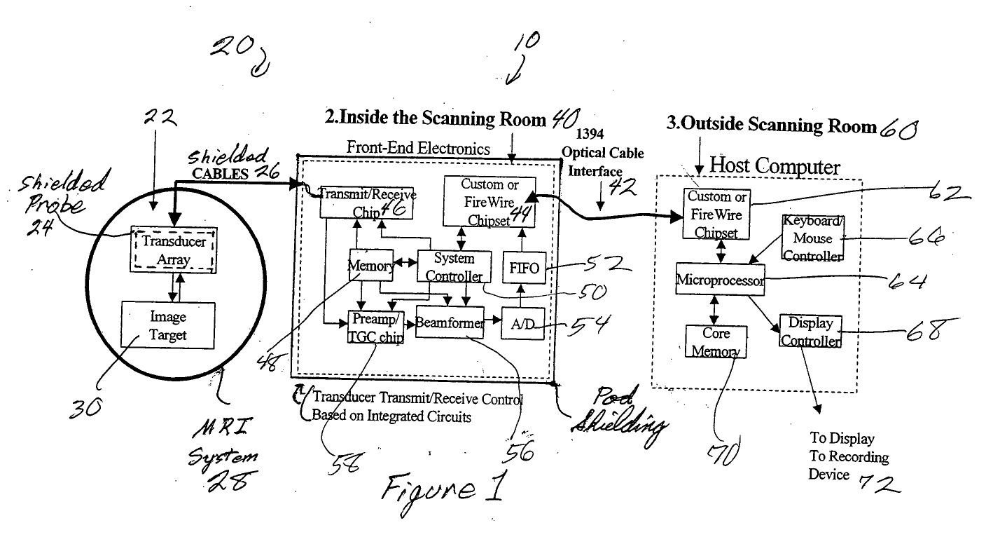

A PC-based system architecture provides a platform for simultaneous MRI Ultrasound imaging applications. As shown in FIG. 1, the system 10 consists of a front-end electronics 40 and a back-end PC as a system controller 60. The front-end integrated electronic chip set is contained in interface housing 40 provides transmit/receive and beamforming functions. Using software implementation, the conventional PC host computer 60 provides imaging formation, baseband conversion, scan conversion, Doppler processing, interface and display control functions. Standard IEEE 1394 FireWire interface 42 provides communication between the front-end electronics and the host computer. As shown in FIG. 1, for simultaneous MRI and ultrasound applications, the transducer probe 24 is placed inside the magnet 28, the front-end electronics is placed inside the scanning room and the host computer is located outside the scanning room. The cable 26 that connects the probe housing to the interface housing 40 is shielded to minimize interference between the MRI and ultrasound systems.

The interface housing 40 includes a transmit and receive circuit 46, a memory 48, a preamplifier and time gain control circuit 58, a beamformer 56 such as a charge domain processor, an analog to digital converter 54, FIFO 52 and system controller 50. A communications interface circuit 44 receives instructions from computer 60 and transmits beamformed image data to computer 60 for further processing. In a preferred embodiment a fiber optical cable 42 is used to connect the front-end electronics and the host PC. For example, Newnex's FireNEX800™ 1394b optical repeater is capable of sending 1394b signals over 1000 meters at speed up to 800 Mbps. Available lengths of Newnex's optical cable for FireNEX800 or other fiber channel device are 10 m , 20 m, 31 m/100′, 50 m, 61 m/200′, 100 m, 122 m/400′, 152 m/500′, 200 m, 500 m, 1000 m. Shielding of the front-end electronics includes the use of special materials such as metal-coated Mylar, like DuPont's MYLAR® XMC110, a polyester film with one side vacuum metallized with aluminum and overcoated with a heat sealable PVdC copolymer. Further details regarding systems and methods for ultrasound imaging can be found in U.S. Pat. Nos. 6,783,493, 6,669,633, 6,111,816, 6,379,304, 6/106,472 and in U.S. application Ser. No. 10/997,062 filed Nov. 24, 2004 and Ser. No. 10/817,316 filed on Apr. 2, 2004, the entire contents of the above patents and applications being incorporated herein by reference.

In a preferred embodiment of the invention, a fixation device or body mounting device can include a corset-like or harness apparatus that fits on the body of the patient, and a fixture thereon that holds an ultrasound transducer. The body mounting device is worn by the patient, for example encircling the chest in the case of a ultrasound and MRI cardiac examination. The technician places an ultrasound transducer within the fixture, and attaches the fixture to the corset. The corset, fixture and transducer are located within the magnetic field, shielded from magnetic and radio frequency interference. The transducer is connected to an electronics interface housing that is also shielded, and the housing is connected to a computer/controller. The housing is preferably located on the floor below the magnet, where the strength of the magnetic flux, lines is reduced compared with inside the magnet. A modification may be made to the special gurney/bed used with the MRI system. The modification consists of removing a section of the pad on the gurney, to allow space for the fixture and transducer when the patient rests on his/her side.

The method consists of manually positioning the transducer before the MRI exam begins. Then, the fixture maintains the transducer at a specific angle and attitude to achieve continuous ultrasound imaging of the appropriate internal organ during the MRI procedure. In one embodiment, a cardiac ultrasound transducer is held in the sub-xyphoid position by the corset/fixture combination, in order to achieve a view of all four chambers of the heart—known as the four chamber view. This view is particularly favorable for continuously monitoring the motion of septum and the left ventricular free wall of the heart, relative to a structure in the chest that is relatively still. In another embodiment, the parasternal short axis view of the heart is chosen to monitor the septum and/or free wall motion from a different perspective. With either of these views, or with other views, tissue Doppler imaging (TDI) can assess the excursion of the septum or free wall and can be used as a surrogate for motion of the heart, enabling the TDI signal to act as a triggering signal for timing of acquisition of MRI images of the heart.

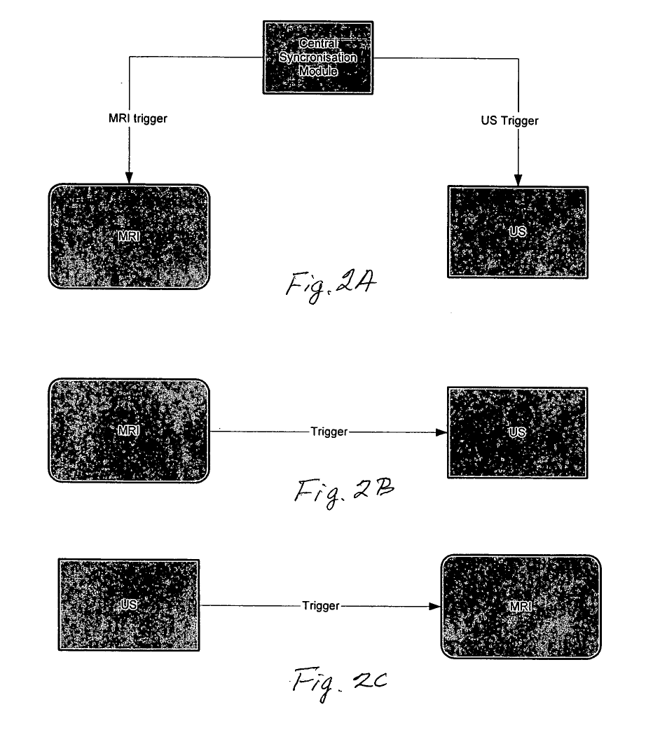

As can be seen in FIG. 2, there are three different methods to achieve simultaneous MRI and ultrasound, Us, image acquisition. FIG. 2A utilizes a central synchronization module to trigger both the MRI and US systems to ensure the receivers of each system are timed to start synchronously. In FIG. 2B, the MRI system sends a trigger signal to the US system to ensure the receivers of the US system are timed to start synchronously with the MRI system. In FIG. 2C, the US system sends a trigger signal to the MRI system to ensure the receivers of the MRI system are timed to start synchronously with the US system.

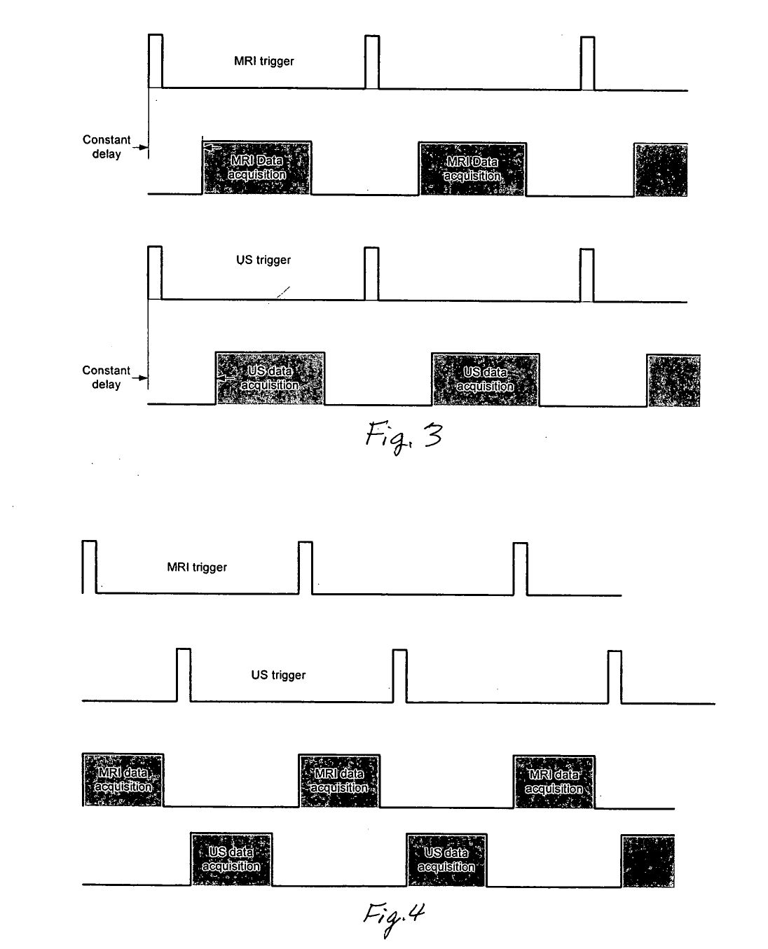

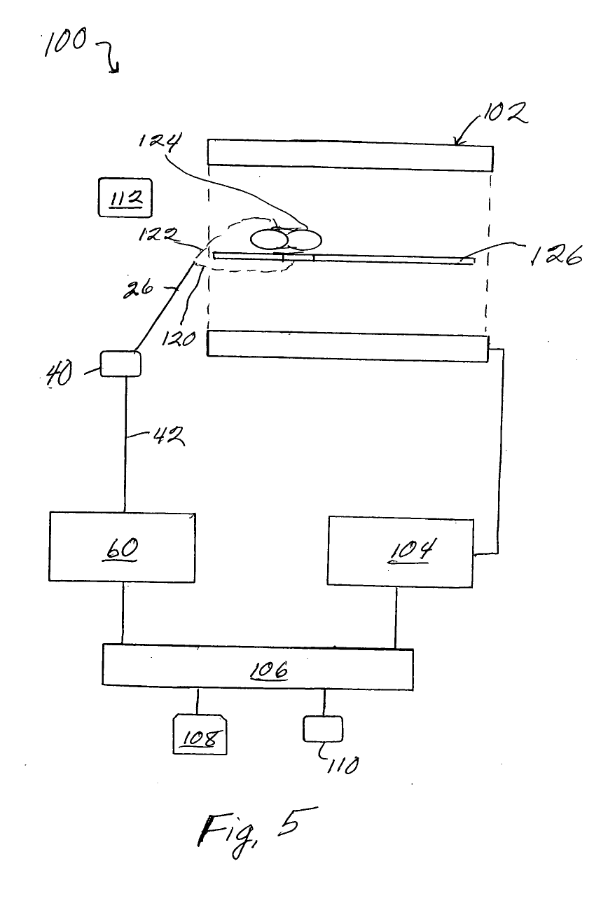

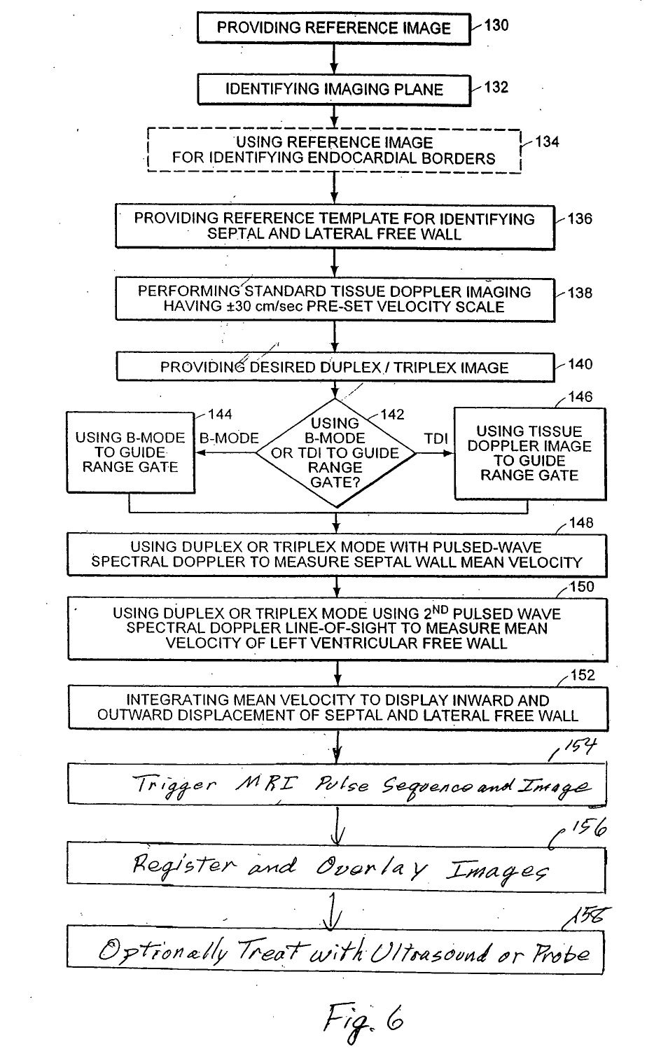

Once the timing of the MRI and US system are synchronous, one can use the trigger signal to gate the image acquisition of the two systems. FIG. 3, illustrates that a synchronized, simultaneously acquisition of both MRI and US images. FIG. 4, illustrates co-registration of non-simultaneously acquisition of both MRI and US images, ie., the trigger signal is used to gate the acquisition of the two systems, however, enough delay is included in the system design to ensure the acquisition of the MRI and US images are sequential and non-overlapped.

Illustrated schematically in FIG. 5 is an integrated imaging system 100 in accordance with a referred embodiment of the invention. The system 100 includes the coil assembly 102 that is actuated by the MRI system 104 that is programmed to control pulse sequence application and MRI image data collection. The ultrasound system including processor 60, interface 40 and probe 24 as shown in FIG. 1 and the MRI processor 104 are both connected to system controller 106 with display 108 and user interface 110. The cable 26 connected to the probe 24 can either be positioned along path 120 to the table 126 or support surface on which a subject lies during the procedure, or along path 122 for positioning on a body mounting device 124 such as a harness or strap. The system can be used to image and/or guide placement of an inventional or probe device 112 such as a catheter, endoscope, biopsy device or needle that is inserted into the tissue, lumen or cavity of a subject.

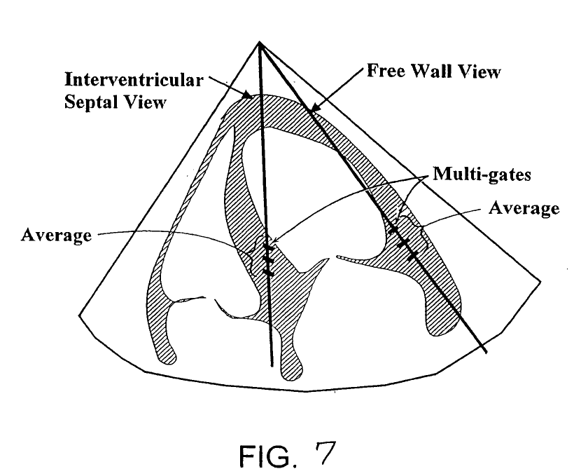

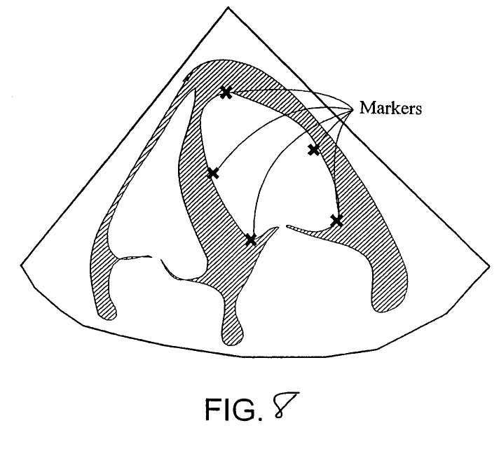

FIG. 6 illustrates an exemplary method for monitoring the synchrony of a heart in accordance with the invention. In the method, a reference template is loaded into memory and used to guide a user in identifying an imaging plane (per step 130). Next a user identifies a desired imaging plane (per step 132). Typically an apical 4-chamber view of the heart is used; however, other views may be used without departing from the spirit of the invention. By way of example, FIG. 7 illustrates an exemplary imaging plane showing the four chambers of a heart.

At times, identification of endocardial borders may be difficult, and when such difficulties are encountered tissue Doppler imaging of the same view may be employed (per step 134). A reference template for identifying the septal and lateral free wall is provided (per step 136). Next, standard tissue Doppler imaging (TDI) with pre-set velocity scales of, say, ±30 cm/sec may be used (per step 138).

Then, a reference of the desired triplex image may be provided (per step 140). Either B-mode or TDI may be used to guide the range gate (per step 142). Alternatively, B-mode can be used for guiding the range gate (per step 144). TDI can be used for guiding the range gate (per step 146). Using TDI or B-mode for guiding the range gate also allows the use of a direction correction angle for allowing the Spectral Doppler to display the radial mean velocity of the septal wall. A first pulsed-wave spectral Doppler is then used to measure the septal wall (or other tissue or organ) mean velocity using duplex or triplex mode (per step 148).

A second range-gate position is also guided using a duplex image or a TDI (per step 150), and a directional correction angle may be used if desired. After step 150, the mean velocity of the septal wall and lateral free wall can be tracked by the system. Time integration of the Spectral Doppler mean velocities at regions of interest (e.g., the septum wall and the left ventricular free wall) then provides the displacement of the septal and left free wall, respectively.

The above method steps may be utilized in conjunction with a high pass filtering means, analog or digital, for removing any baseline disturbance present in collected signals. In addition, the disclosed method can employ multiple simultaneous PW Spectral Doppler lines for tracking movement of the interventricular septum and the left ventricular free wall. In addition, a multiple gate structure may be employed along each spectral line, thus allowing quantitative measurement of regional wall motion. Averaging over multiple gates may allow measurement of global wall movement, which can be seen in FIG. 7. The inward and outward displacement of the septal and lateral free wall is then displayed by way of integrating the mean velocity (per step 152). The data obtained can be used to actuate or trigger an MRI pulse sequence and obtain one or more MRI images 154. This can be followed by registration and/or overlay 156 of the two images. The system can also be used for ultrasound treatment 158 in certain applications.

An exemplary method based on B-mode image data sequences and a motion-compensated block-match image enhancement technique can be used to detect the boundary. A B-mode cine loop is an ordered collection of still frames that display sequential “snapshots” of the beating heart as viewed from a particular imaging plane. Thus within a given cine loop, most of the variation in intensity from one frame to the next is due to object motion. Motion-Compensated (MC) image processing refers to mathematical analysis of a sequence of images to account for translation (and possible rotation and deformation) of objects within the field of view.

The method begins by initializing the phase analyses and motion tracking application. An initial B-mode image frame is received. The image frame is then segmented into multi-pixel blocks. A user then marks a set of anchor points on a displayed image using an input device such as a computer mouse, stylus, or keyboard. To minimize the computation requirement by reducing the search area, a user can manually place a plurality of, for example 5, “anchor points” along the endocardial border of left ventricle, as shown in FIG. 8. This can be used to register the different images of the heart, for example a phantom can also be used to provide registration of the ultrasound image and the MRI image.

The claims should not be read as limited to the described order or elements unless stated to that effect. Therefore, all embodiments that come within the scope and spirit of the following claims and equivalents thereto are claimed as the invention.

1. A medical imaging system comprising:
an ultrasound imaging system;
a magnetic resonance imaging system; and
a controller connected to the ultrasound imaging system and the magnetic resonance imaging (MRI) system, the controller actuating synchronized acquisition of an ultrasound image and an MRI image.

2. The system of claim 1 further comprising a shielded ultrasound transducer probe.

3. The system of claim 2 further comprising a shielded interface housing connected to the transducer probe and a processing system, the interface housing having a beamformer circuit, and a cable connecting the interface housing to the processing system.

4. The system of claim 1 wherein the controller comprises a timing system to control an acquisition sequence of an ultrasound image and an MRI image.

5. The system of claim 3 wherein a fiber optic cable connects the interface housing and the processing system.

6. The system of claim 1 wherein the ultrasound imaging system further comprises a probe positioned within a coil assembly of the MRI system.

7. The system of claim 1 wherein the controller synchronously operates the ultrasound imaging system and the MRI imaging system.

8. The system of claim 7 wherein the controller actuates synchronized simultaneous acquisition of an ultrasound image and an MRI image.

9. The system of claim 7 wherein the controller actuates synchronous gated acquisition of ultrasound data and MRI data.

10. The system of claim 1 wherein the interface housing is connected to a computer using a standard communications interface.

11. The system of claim 1 further comprising a body mounting device with which an ultrasound transducer probe can be mounted to a body of a subject.

12. The system of claim 11 wherein the body mounting device comprises a harness.

13. The system of claim 12 wherein the harness positions the probe relative to a heart of the subject.

14. The system of claim 1 further comprising a support surface on which a subject is positioned relative to a coil assembly of the MRI system.

15. The system of claim 1 further comprising a probe device for insertion into a subject.

16. The system of claim 15 wherein the probe device comprises a catheter, such that a position of the catheter in a body of the subject can be imaged.

17. The system of claim 15 wherein the probe device comprises an endoscope.

18. The system of claim 15 wherein the probe device comprises a biopsy device.

19. The system of claim 1 wherein the MRI system is programmed to generate a navigator signal.

20. The system of claim 1 wherein the system is programmed to perform Doppler imaging of a region of interest.

21. The system of claim 1 wherein the ultrasound image and the MRI image are registered.

22. The system of claim 21 wherein the ultrasound image and the MRI image can be overlayed on a display device.

23. The system of claim 3 wherein the interface housing has an interference shield.

24. The system of claim 3 wherein the interface housing has an IEEE 1394 standard communication circuit that transmits beamformed data.

25. The system of claim 3 wherein the interface housing further houses a memory circuit and an interface controller circuit connected to the beamformer circuit.

26. The system of claim 3 wherein the interface housing is connected to the transducer probe with a shielded cable.

27. The system of claim 3 wherein the interface housing further houses a transmit and receive circuit that communicates with the transducer probe, and further houses a preamplifier circuit and time gain compensation (TGC) circuit.

28. The system of claim 3 wherein the processing system includes a processor that performs scan conversion and Doppler processing.

29. The system of claim 3 wherein the processing system further comprises a memory and a standard communication interface that receives and transmits signals to the interface housing.

30. The system of claim 1 wherein an ultrasound transducer probe is positioned for cardiac imaging.

31. The system of claim 1 wherein the ultrasound image is a spectral Doppler image.

32. The system of claim 1 wherein the ultrasound probe acquires a three-dimensional image of a volume of interest.

33. The system of claim 1 wherein an ultrasound transducer probe is positioned for brain imaging.

34. The system of claim 1 wherein the ultrasound system performs a treatment operation on a region of interest.

35. The system of claim 34 wherein the treatment operation comprises a thermal treatment with MRI monitoring of temperature of the region of interest.

36. A method for imaging a regional interest comprising:
positioning a transducer probe relative to a subject, the transducer probe being connected to an ultrasound imaging system;
positioning the subject and the transducer probe within a coil assembly of a magnetic resonance imaging (MRI) system; and
obtaining an ultrasound image and an MRI image of a region of interest within the subject using a controller connected to the ultrasound imaging system and the magnetic resonance imaging system.

37. The method of claim 36 further comprising providing a shielded ultrasound transducer probe.

38. The method of claim 36 further comprising providing a shielded interface housing connected to the transducer probe and a processing system, the interface housing having a beamformer circuit and a fiber optic connection to the processing system.

39. The method of claim 36 further comprising actuating a timing system to control an acquisition sequence.

40. The method of claim 36 further comprising registering the ultrasound image and the MRI image.

41. The method of claim 36 wherein the MRI system triggers an ultrasound image acquisition.

42. The method of claim 36 wherein the ultrasound system triggers an MRI image acquisition.

43. The method of claim 39 wherein the acquisition of MRI data and ultrasound data is synchronous.

44. The method of claim 36 wherein the ultrasound image and MRI image are overlayed on a display.

45. The method of claim 39 wherein the acquisition of MRI data and ultrasound data is synchronous and simultaneous.

46. The method of claim 36 further comprising using navigator MRI signals to identify a moving object to be imaged.

47. The method of claim 36 further comprising performing treatment on a region of interest with the ultrasound imaging system.

48. The method of claim 47 further comprising thermally treating the region of interest with the ultrasound system.

49. The method of claim 48 further comprising monitoring the treatment with the MRI system.

50. A method of registering ultrasound and magnetic resonance images comprising
registering an ultrasound image with a magnetic resonance (MR) image; and
overlaying the ultrasound image and the MR image.

51. The method of claim 50 wherein the images are cardiac images.

52. The method of claim 50 wherein the ultrasound image is a Doppler image.
